(19) United States
(12) Patent Application Publication
(10) Pub. No.: US 2025/0170129 A1
(43) Pub. Date: May 29, 2025

(54) SENOTHERAPEUTIC AGENTS AND ALPHA-KLOTHO POLYPEPTIDES

(71) Applicant: Mayo Foundation for Medical Education and Researc, Rochester, MN (US)

(72) Inventors: Yi Zhu, Rochester, MN (US); James L. Kirkland, Rochester, MN (US); Tamar Tchkonia, Rochester, MN (US); Nino Giorgadze, Rochester, MN (US); Tamar Pirtskhalava, Rochester, MN (US)

(21) Appl. No.: 18/840,279

(22) PCT Filed: Mar. 3, 2023
(86) PCT No.: PCT/US2023/014452

§ 371 Date: Aug. 21, 2024

Related U.S. Application Data
(60) Provisional application No. 63/317,744, filed on Mar. 8, 2022.

(51) Int. Cl. A61K 31/506; A61K 31/352; A61P 43/00; C07K 16/22; C07K 16/24; G01N 33/68

(52) U.S. Cl. CPC A61K 31/506; A61K 31/352; A61P 43/00; C07K 16/22; C07K 16/245; G01N 33/6893; G01N 2333/924

(57) ABSTRACT

This document relates to methods and materials for assessing and/or using one or more senotherapeutic agents. In some cases, methods and materials for determining the efficacy of an anti-senescence treatment in a mammal (e.g., a human) are provided. For example, a level of one or more α-Klotho polypeptides in a sample (e.g., a urine sample) from a mammal (e.g., a human) can be used to determine the efficacy of the one or more senotherapeutic agents. In some cases, methods and materials for treating a mammal (e.g., a human) having a disease or disorder characterized by a reduced level of an α-Klotho polypeptide are provided. For example, one or more senotherapeutic agents and/or one or more inhibitors of a senescence-associated secretory phenotype (SASP) polypeptide can be administered to a mammal (e.g., a human) to increase a level of α-Klotho polypeptides within the mammal.

### CROSS-REFERENCE TO RELATED APPLICATIONS

This application claims the benefit of U.S. Patent Application Ser. No. 63/317,744, filed on Mar. 8, 2022. The disclosure of the prior application is considered part of (and is incorporated by reference in) the disclosure of this application.

### STATEMENT REGARDING FEDERAL FUNDING

This invention was made with government support under AG013925, AG062413, AG061456, AG044271, AG013319, and AG021332 awarded by the National Institutes of Health. The government has certain rights in the invention.

### TECHNICAL FIELD

This document relates to methods and materials for assessing and/or using one or more senotherapeutic agents. In some cases, the methods and materials provided herein can be used to determine the efficacy of an anti-senescence treatment in a mammal (e.g., a human). For example, a level of one or more alpha (α)-Klotho polypeptides in a sample (e.g., a urine sample) obtained from a mammal (e.g., a human) having been administered one or more senotherapeutic agents can be used to determine the efficacy of the one or more senotherapeutic agents. In some cases, this document provides methods and materials for treating a mammal (e.g., a human) having a disease or disorder characterized by a reduced level of an α-Klotho polypeptide. For example, one or more senotherapeutic agents and/or one or more inhibitors of a senescence-associated secretory phenotype (SASP) polypeptide can be administered to a mammal (e.g., a human) to increase a level of α-Klotho polypeptides within the mammal.

### BACKGROUND INFORMATION

α-Klotho is a geroprotective polypeptide that exerts anti-physiological stress effects (Maique et al., Front. Pharmacol., 11:1273 (2020)) and protects against oxidative damage, hypoxia, and cytotoxic drugs (Kuro, Nat. Rev. Nephrol., 15(1):27-44 (2019); and Kuro, Nephrol. Dial. Transplant., 34(1):15-21 (2019)). Several preclinical studies have implicated α-Klotho polypeptide as a molecule that impacts lifespan, health-span, and renal and cognitive function (Kuro, Nat. Rev. Nephrol., 15(1):27-44 (2019); and Cheikhi et al., J. Gerontol. A Biol. Sci. Med. Sci., 74(7):1031-42 (2019)). As the α-Klotho polypeptide holds therapeutic potential, various strategies to increase α-Klotho polypeptide expression have been proposed. However, despite extensive effort there are few approaches for increasing or restoring α-Klotho polypeptide expression that are feasible or that have shown efficacy in trials.

### SUMMARY

This document provides methods and materials for assessing and/or using one or more senotherapeutic agents. In some cases, the methods and materials provided herein can be used to determine the efficacy of an anti-senescence treatment in a mammal (e.g., a human). For example, a level of one or more α-Klotho polypeptides in a sample (e.g., a urine sample) obtained from a mammal (e.g., a human) having been administered one or more senotherapeutic agents can be used to determine the efficacy of the one or more senotherapeutic agents. In some cases, this document provides methods and materials for treating a mammal (e.g., a human) having a disease or disorder characterized by a reduced level of an α-Klotho polypeptide. For example, one or more senotherapeutic agents and/or one or more inhibitors of a SASP polypeptide can be administered to a mammal (e.g., a human) to increase a level of α-Klotho polypeptides within the mammal.

As demonstrated herein, the presence of senescent cells can reduce the level α-Klotho polypeptides, and the level of α-Klotho polypeptides in a sample (e.g., a urine sample) obtained from a mammal can be used to determine the efficacy of an anti-senescence treatment (e.g., one or more senotherapeutic agents) in that mammal. Also as demonstrated herein, administering one or more senotherapeutic agents to a mammal (e.g., a human) can increase α-Klotho expression within the mammal. For example, one or more senotherapeutic agents can be administered to a mammal (e.g., a human) having a disease or disorder characterized by a reduced level of an α-Klotho polypeptide (e.g., idiopathic pulmonary fibrosis (IPF)) to treat the mammal.

In general, one aspect of this document features methods for assessing efficacy of an anti-senescence treatment. The methods can include, or consist essentially of, (a) detecting a level of an α-Klotho polypeptide in a first urine sample obtained from a mammal prior to or within 24 hours of administration of a anti-senescence treatment to the mammal; (b) detecting a level of the α-Klotho polypeptide in a second urine sample obtained from the mammal at least 120 hours after administration of the anti-senescence treatment to the mammal; (c) identifying the anti-senescence treatment as being effective if the level of the α-Klotho polypeptide in the second urine sample is greater than the level of the α-Klotho polypeptide in the first urine sample; and (d) identifying the anti-senescence treatment as being not effective if the level of the α-Klotho polypeptide in the second urine sample is less than or equal to the level of the α-Klotho polypeptide in the first urine sample. The mammal can be a human. The first urine sample can be obtained prior to the mammal having been administered the anti-senescence treatment. The first urine sample can be obtained after the mammal has been administered the anti-senescence treatment.

In another aspect, this document features methods for assessing efficacy of an anti-senescence treatment. The methods can include, or consist essentially of, detecting a level of an α-Klotho polypeptide in a urine sample obtained from a mammal at least 120 hours after administration of an anti-senescence treatment to the mammal, where the anti-senescence treatment is identified as being effective if the level of the α-Klotho polypeptide in the sample is greater than 293.49±115.48 ng of the α-Klotho polypeptide per mg of creatinine present in the urine sample, and where the anti-senescence treatment is identified as being ineffective if the level of the α-Klotho polypeptide in the sample is less than 293.49±115.48 ng of the α-Klotho polypeptide per mg of creatinine present in the urine sample. The mammal can be a human.

In another aspect, this document features methods for increasing a level of an α-Klotho polypeptide in a mammal. The methods can include, or consist essentially of, administering a senotherapeutic agent to a mammal. The mammal can be a human. The human can be identified as being in need of increased α-Klotho polypeptide expression. The mammal can have fibrosis (e.g., IPF). The senotherapeutic agent can be dasatinib, quercetin, navitoclax, A1331852, A1155463, fisetin, luteolin, geldanamycin, tanespimycin, alvespimycin, piperlongumine, panobinostat, FOX04-related peptides, nutlin3a, ruxolitinib, metformin, rapamycin procyanidin C1, SSK1, Prodrug A (JHB75B), 5FURGal, Nav-Gal, PZ15227, PROTAC ARV825, or CD9-Lac/CaCO3/Rapa nanoparticles. The level of the α-Klotho polypeptide can be detected in a urine sample obtained from the mammal. The level of the α-Klotho polypeptide can be greater than 293.49±115.48 ng of the α-Klotho polypeptide per mg of creatinine present in the urine sample.

In another aspect, this document features methods for increasing a level of an α-Klotho polypeptide in a mammal. The methods can include, or consist essentially of, administering an inhibitor of a SASP polypeptide to a mammal. The mammal can be a human. The human can be identified as being in need of increased α-Klotho polypeptide expression. The mammal can have fibrosis (e.g., IPF). The SASP polypeptide can be an activin A polypeptide or an interleukin 1α (IL-1α) polypeptide. The inhibitor of the SASP polypeptide can be a neutralizing antibody. The level of the α-Klotho polypeptide can be detected in a urine sample obtained from the mammal. The level of the α-Klotho polypeptide can be greater than 293.49±115.48 ng of the α-Klotho polypeptide per mg of creatinine present in the urine sample.

In another aspect, this document features uses of a composition including a senotherapeutic agent to increase α-Klotho polypeptide expression in a mammal. The mammal can be a human. The human can have fibrosis. The level of the α-Klotho polypeptide can be detected in a urine sample obtained from the mammal. The level of the α-Klotho polypeptide can be greater than 293.49±115.48 ng of the α-Klotho polypeptide per mg of creatinine present in the urine sample.

In another aspect, this document features uses of a composition comprising an inhibitor of a SASP polypeptide to increase α-Klotho polypeptide expression in a mammal. The mammal can be a human. The human can have fibrosis. The level of the α-Klotho polypeptide can be detected in a urine sample obtained from the mammal. The level of the α-Klotho polypeptide can be greater than 293.49±115.48 ng of the α-Klotho polypeptide per mg of creatinine present in the urine sample.

In another aspect, this document features a senotherapeutic agent for use in the preparation of a medicament to increase α-Klotho polypeptide expression in a mammal. The mammal can be a human. The human can have fibrosis. The level of the α-Klotho polypeptide can be detected in a urine sample obtained from the mammal. The level of the α-Klotho polypeptide can be greater than 293.49±115.48 ng of the α-Klotho polypeptide per mg of creatinine present in the urine sample.

In another aspect, this document features a senotherapeutic agent for use in increasing α-Klotho polypeptide expression in a mammal. The mammal can be a human. The human can have fibrosis. The level of the α-Klotho polypeptide can be detected in a urine sample obtained from the mammal. The level of the α-Klotho polypeptide can be greater than 293.49±115.48 ng of the α-Klotho polypeptide per mg of creatinine present in the urine sample.

In another aspect, this document features an inhibitor of a SASP polypeptide for use in the preparation of a medicament to increase α-Klotho polypeptide expression in a mammal. The mammal can be a human. The human can have fibrosis. The level of the α-Klotho polypeptide can be detected in a urine sample obtained from the mammal. The level of the α-Klotho polypeptide can be greater than 293.49±115.48 ng of the α-Klotho polypeptide per mg of creatinine present in the urine sample.

In another aspect, this document features an inhibitor of a SASP polypeptide for use in increasing α-Klotho polypeptide expression in a mammal. The mammal can be a human. The human can have fibrosis. The level of the α-Klotho polypeptide can be detected in a urine sample obtained from the mammal. The level of the α-Klotho polypeptide can be greater than 293.49±115.48 ng of the α-Klotho polypeptide per mg of creatinine present in the urine sample.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIGS. 1A-1F. Senescent cell conditioned medium decreases α-Klotho; blocking particular SASP factors with neutralizing antibodies partially restores α-Klotho. FIGS. 1A-1C) Conditioned media (CM) collected from senescent or non-senescent (FIG. 1A) human primary kidney endothelial cells (HKEs; one-way ANOVA: F=19.10, p<0.0001, R²=0.89; n=3; ****p<0.0001); (FIG. 1B) human umbilical vein endothelial cells (HUVECs; one-way ANOVA: F=3.02, p=0.02, R²=0.58, n=3; *p=0.02, **p=0.01); or (FIG. 1C) human brain astrocytes (HBAs; one-way ANOVA: F=7.93, p=0.02, R²=0.66; n=5; ***p<0.001, *p=0.03) were applied to non-senescent HKEs, HUVECs, or HBAs, respectively, with or without antibodies against PAI-1, CXCL1, IL-6, activin A, TNF-α, IL-1α, IL-1β, or IL-18 for 48 hours. α-Klotho mRNA was assayed by qPCR. Means SEM. FIGS. 1D-1F) Primary HKEs (FIG. 1D; n=4), HUVECs (FIG. 1E; n=4), or HBAs (FIG. 1F; n=4) were treated with recombinant activin A or IL-1α. α-Klotho was assayed by qPCR 24 hours after treatments. Means±SEM; unpaired T-tests; ***p<0.001, ****p<0.0001.

FIGS. 2A-2F. Transplanting senescent cells decreases urinary and brain α-Klotho. FIG. 2A) 8-month-old male mice were transplanted i.p. with 2 million senescent or non-senescent mouse preadipocytes in 150 μL PBS through a 22-G needle. FIG. 2B) Urinary α-Klotho was assayed by ELISA and expressed as a function of creatinine (one-way ANOVA: F=6.45, p=0.01, R²=0.42; n=7; **p<0.01, *p=0.02). Representative images of immunofluorescent (IF) α-Klotho stains of cerebellum (FIG. 2C) and choroid plexus (FIG. 2E) are shown. ROI (Region of Interest, outlined in white) were quantified by ImageJ (FIGS. 2D and 2F) from mice that had been transplanted with senescent or non-senescent cells. Means±SEM; one-way ANOVA and post hoc Tukey's tests: F=4.94, p=0.02, R²=0.38. *p=0.02, *p=0.03 in (FIG. 2D), F=7.84; p<0.01, R²=0.38, **p<0.01, *p=0.01 in (FIG. 2F).

FIGS. 3A-3I. Genetic clearance of highly p16^{Ink4a}-expressing cells increases α-Klotho. Young (8-month-old) or old (27-29-month-old) INK-ATTAC male mice were treated with vehicle or AP20187 (n=3 young+vehicle; n=3 young+AP20187; n=7 old+vehicle; n=7 old+AP20187) to dimerize the FKBP-caspase-8 fusion protein expressed in highly p16^{Ink4a}-expressing cells to selectively eliminate p16^{Ink4a-} cells. AP20187 (10 mg/kg) or vehicle was administered i.p. every 2 weeks for 6 weeks. FIG. 3A) Schematic view of treatments. FIG. 3B) Kidney α-Klotho mRNA was assayed by qPCR. One-way ANOVA, F=4.81, p=0.01, R²=0.46; *p=0.03, **p<0.01. FIG. 3C) α-Klotho protein was assayed relative to GAPDH (Western blots in FIG. 13). One-way ANOVA: F=4.81, p=0.01, R²=0.46; *p<0.05, ****p<0.0001. FIG. 3D) Mouse urine was collected 2 days after the last dose of AP20187. Urinary α-Klotho was assayed by ELISA and expressed as a function of creatinine. One-way ANOVA, F=61.53, p<0.00001, R²=0.90; *p=0.04, ****p<0.0001. Choroid Plexus (FIG. 3E) α-Klotho protein was assayed by immunofluorescence. One-way ANOVA, F=20.98, R²=0.80, **p<0.01, ****p<0.0001. Hippocampal (FIG. 3F) and cerebellar (FIG. 3G) α-Klotho mRNA was assayed by qPCR. Activin A mRNA in kidney (FIG. 3H) and IL-1α mRNA in hippocampus (FIG. 3I) were assayed by qPCR. Means±SEM; one-way ANOVA and post hoc Tukey's tests; F=3.84, p=0.038, R²=0.40; *p=0.04 in (FIG. 3F); *p=0.04, F=7.87, p<0.01, R²=0.60; *p=0.03 in (FIG. 3G); F=10.85; p<0.001, R²=0.64; *p=0.03, ***p<0.0001 in (FIG. 3H); F=5.49; p=0.01, R²=0.49; *p<0.05, **p=0.01 in (FIG. 3I).

FIGS. 4A-4D. Senolytics increase urinary and kidney α-Klotho in old or obese mice. FIG. 4A) Naturally-aged male mice (28-29-month-old, n=10 in each group) were treated with vehicle, Dasatinib plus Quercetin (D+Q), or Fisetin. Urinary α-Klotho was measured by ELISA and expressed as a function of creatinine. One-way ANOVA: F=6.39; p<0.0⁵, R²=0.30; *p=0.02, **p<0.01. FIG. 4B) DIO mice (male, 10-month-old, n=8) were treated with vehicle or D+Q and urinary α-Klotho was assayed by ELISA and expressed as a function of creatinine. Unpaired T test; **p<0.01. FIG. 4C) Six-month-old male mice (n=6 in each group) were transplanted i.p. with senescent preadipocytes. After 3 months, they were treated with vehicle, D+Q, or Fisetin. Urinary α-Klotho was assayed by ELISA and expressed as a function of creatinine. One-way ANOVA and post hoc Tukey's tests: Kruskal-Wallis statistic=10.19; *p=0.02, **p<0.01. FIG. 4D) Kidney α-Klotho protein was also assayed in the same transplanted mice by Western blotting. One-way ANOVA: F=24.24; p<0.001, R²=0.83; ***p<0.001.

FIGS. 5A-5E. Senolytics increase brain α-Klotho in old mice. Naturally-aged mice (female, 28-month-old) were treated with vehicle (n=5) or D+Q (n=5). Brain α-Klotho was analysed by IF. FIG. 5A) Representative IF images of α-Klotho expression in the cerebellum are shown. Mean intensities of florescence in the cerebellum were quantified by ImageJ. Unpaired T-tests; *p=0.03. FIG. 5B) Brain choroid plexus α-Klotho, representative IF images. Mean intensities of florescence in the choroid plexus were quantified by ImageJ. Unpaired T-tests; *p=0.02. FIG. 5C) Young (female, 6-month-old, n=5 in vehicle and treated groups) and naturally-aged mice (female, 22-month-old, n=8 in vehicle and treated groups) were treated with Fisetin or vehicle. α-Klotho in whole brain was assayed by qPCR. One-way ANOVA and post hoc Tukey's tests: Kruskal-Wallis statistic=9.49; *p=0.02, **p<0.01. FIG. 5D) DIO mice (male, 8-9-month-old) were treated with D+Q (n=8) or vehicle (n=8). Brain α-Klotho was assayed by qPCR. Mann-Whitney test; *p=0.03. FIG. 5E) Correlation between brain α-Klotho mRNA and peripheral p16^{Ink4a}-expressing adipose tissue progenitor cells was quantified by CyTOF. Spearman correlation analysis.

FIGS. 6A-6D. Senolytics increase α-Klotho in human urine; urinary SASP factors are inversely related to urinary α-Klotho. Subjects (n=20) with idiopathic pulmonary fibrosis (IPF) were administered 3 courses of D+Q for 3 sequential days each (total 9 doses over 3 weeks). FIG. 6A) Urinary α-Klotho and SASP factors were assayed at baseline and 5 days after the last D+Q treatment. Urinary α-Klotho in subjects with IPF was increased (Baseline: 293.49±115.48, Post-treatment: 392.79±95.24, normalized to urinary creatinine) by D+Q. Wilcoxon signed rank tests (two-sided) were used to test differences before and after treatment. *p=0.04. FIGS. 6B-6D) Spearman correlations of urinary α-Klotho and SASP factors are shown. FDR-corrected R² and p values are in Table 2.

FIG. 7. Heatmap of senescence markers and SASP factors. Human primary kidney endothelial cells (n=3), HUVECs (n=3), or human brain astrocytes (n=3) were radiated (10 Gy) to generate senescence. 20 days later, senescence and SASP markers were analysed by qPCR. Mean relative expression is illustrated in the heat map, and fold changes are depicted in the colour key in the lane to the right.

FIG. 8. Senescent cell conditioned medium decreases α-Klotho; blocking particular SASP factors with neutralizing antibodies partially restores α-Klotho. CM collected from senescent or non-senescent HBAs (n=3) was applied to non-senescent HBAs, respectively, with or without antibodies against IL-1α and Activin A for 48 hours. α-Klotho protein was assayed by immunofluorescence staining and quantified by ImageJ. Means±SEM. One-way ANOVA, F=8.81, R²=0.66; *p=0.017, **p=0.0067.

FIGS. 9A-9C. Kidney α-Klotho declines with ageing; urinary α-Klotho declines with ageing and in obesity. FIG. 9A) α-Klotho in kidneys of young (3-month-old) and old (28-month-old) mice were assayed by Western blotting. GAPDH was used as a loading control. FIG. 9B) Urinary α-Klotho of young (6-month-old) and old (24-month-old) mice was assayed by ELISA and expressed as a function of creatinine. FIG. 9C) Urinary α-Klotho in lean (12-month-old) and DIO (12-month-old) mice was assayed by ELISA and expressed as a function of creatinine.

FIGS. 10A-10B. Genetic clearance of highly p16^{Ink4a}-expressing cells and senolytics increase urinary α-Klotho in old mice. Old (25-26-month-old) INK-ATTAC male mice were treated with vehicle or AP20187 or D+Q (n=7). AP20187 or D+Q or vehicle was administered every 2 weeks for 6 weeks. Mouse urine was collected 5 days after the last dose of AP20187 or D+Q. Urinary α-Klotho was assayed by ELISA and expressed as a function of creatinine (FIG. 10A) or Cystatin C (FIG. 10B). One-way ANOVA, A: *p=0.038, **p=0.0066, B: *p=0.048, **p=0.0048.

FIGS. 11A-11C. Senolytic compounds do not transcriptionally up-regulate α-Klotho in non-senescent cells. Non-senescent human astrocytes were treated with vehicle, Dasatinib (200 nM) plus Quercetin (10 μM), or Fisetin (10 μM) for 48 hours. FIG. 11A) α-Klotho was assayed by Western blotting with GAPDH as the loading control. FIG. 11B) α-Klotho mRNA was assayed by qPCR. FIG. 11C) Non-senescent human adipose progenitor cells were treated with Dasatinib (500 nM) plus Quercetin (15 M) or vehicle for 48 hours. α-Klotho mRNA was assayed by qPCR.

FIGS. 12A-12B. Senolytic compounds do not transcriptionally up-regulate α-Klotho in young animals. 3-month-old female INK-ATTAC mice were treated with vehicle, Dasatinib (5 mg/kg) plus Quercetin (50 mg/kg), or AP20187 (10 mg/kg). FIG. 12A) Kidneys were harvested and lysed to assay α-Klotho protein by Western blotting. FIG. 12B) Urinary α-Klotho in the same cohort was assayed by ELISA after a second and third treatment with senolytic agents and expressed as a function of creatinine.

FIG. 13. Decreasing highly p16^{Ink4a}-expressing cells increases α-Klotho. See FIG. 3D. Young (8-month-old) or old (27-29-month-old) INK-ATTAC male mice were treated with vehicle or AP20187 (n=3; young; n=7 old+vehicle; n=7 old+AP20187) to dimerize the FKBP-caspase-8 fusion to selectively eliminate highly p16^{Ink4a}-expressing cells. AP20187 (10 mg/kg) or vehicle was administered i.p. every 2 weeks for 6 weeks. Western blot images of α-Klotho and GAPDH in the kidneys of young, old, and AP20187-treated INK-ATTAC mice are shown.

FIG. 14. Senolytics can reduce cellular senescence and tissue dysfunction, and can increase expression of a α-Klotho polypeptide.

### DETAILED DESCRIPTION

This document provides methods and materials for assessing and/or using one or more senotherapeutic agents. In some cases, the methods and materials provided herein can be used to determine the efficacy of an anti-senescence treatment in a mammal (e.g., a human). For example, a level of one or more α-Klotho polypeptides in a sample (e.g., a urine sample) obtained from a mammal (e.g., a human) having been administered one or more senotherapeutic agents can be used to determine the efficacy of the one or more senotherapeutic agents. In some cases, this document provides methods and materials for treating a mammal (e.g., a human) having a disease or disorder characterized by a reduced level of an α-Klotho polypeptide. For example, one or more senotherapeutic agents and/or one or more inhibitors of a SASP polypeptide can be administered to a mammal (e.g., a human) to increase a level of α-Klotho polypeptides within the mammal.

Any appropriate mammal can be assessed and/or treated as described herein. Examples of mammals that can be assessed and/or treated as described herein include, without limitation, humans, non-human primates such as monkeys, dogs, cats, horses, cows, pigs, sheep, mice, rats, hamsters (e.g., Syrian hamsters), and rabbits.

A mammal (e.g., a human) having been administered an anti-senescence treatment (e.g., having been administered one or more senotherapeutic agents) can be assessed to determine efficacy of the anti-senescence treatment by detecting the presence, absence, or level of one or more α-Klotho polypeptides in a sample (e.g., a urine sample) obtained from the mammal. As described herein, senescent cells can cause a reduction in a level of α-Klotho polypeptides produced by cells within a mammal (e.g., a human) that can be detected in that mammal's urine. Accordingly, urinary levels of one or more α-Klotho polypeptides can be used to determine whether the number of senescent cells in a mammal (e.g., a human) are increasing, decreasing, or staying essentially the same, and can therefore be used to determine whether an anti-senescence treatment is effective.

In some cases, a sample (e.g., a urine sample) can be obtained from a mammal (e.g., a human) having been administered an anti-senescence treatment (e.g., having been administered one or more senotherapeutic agents). When a level of one or more α-Klotho polypeptides in a urine sample obtained from a mammal after the mammal has been administered an anti-senescence treatment (e.g., one or more senotherapeutic agents) is greater than 293.49±115.48 ng of α-Klotho polypeptides per mg creatinine, the anti-senescence treatment (e.g., one or more senotherapeutic agents) can be determined to be an effective treatment for that mammal. For example, when a level of one or more α-Klotho polypeptides in a urine sample obtained from a mammal after the mammal has been administered an anti-senescence treatment (e.g., one or more senotherapeutic agents) is from about 295 ng of α-Klotho polypeptides per mg creatinine to about 500 ng of α-Klotho polypeptides per mg creatinine (e.g., 392.79±95.24), the anti-senescence treatment (e.g., one or more senotherapeutic agents) can be determined to be an effective treatment for that mammal. When a level of one or more α-Klotho polypeptides in a urine sample obtained from a mammal after the mammal has been administered an anti-senescence treatment (e.g., one or more senotherapeutic agents) is less than or equal to 293.49±115.48 ng of α-Klotho polypeptides per mg creatinine, the anti-senescence treatment (e.g., one or more senotherapeutic agents) can be determined to not be an effective treatment for that mammal.

A sample (e.g., a urine sample) can be obtained from a mammal (e.g., a human) at any time after the mammal has been administered an anti-senescence treatment (e.g., one or more senotherapeutic agents). In some cases, a urine sample can be obtained from mammal (e.g., a human) within 24 hours (e.g., within 12 hours) of the mammal having been administered an anti-senescence treatment (e.g., one or more senotherapeutic agents). In some cases, a urine sample can be obtained from mammal (e.g., a human) at least 120 hours after the mammal has been administered an anti-senescence treatment (e.g., one or more senotherapeutic agents).

In certain instances, a level of one or more α-Klotho polypeptides within a mammal (e.g., within a sample obtained from a mammal) can be detected at different time points over a course of an anti-senescence treatment to determine efficacy of the anti-senescence treatment. For example, two or more (e.g., two, three, four, five, six, or more) urine samples can be obtained from a mammal at different time point over the course of an anti-senescence treatment, and the level of one or more α-Klotho polypeptides in the sample can be used to determine the efficacy of the anti-senescence treatment.

In some cases, a first sample (e.g., a first urine sample) can be obtained from a mammal (e.g., a human) prior to the mammal being administered an anti-senescence treatment (e.g., prior to being administered one or more senotherapeutic agents), and a second sample (e.g., a second urine sample), and optionally subsequence samples, can be obtained from the mammal after the mammal has been administered the anti-senescence treatment. When a level of one or more α-Klotho polypeptides in the first sample is greater than the level of the α-Klotho polypeptide(s) in the second sample, the anti-senescence treatment (e.g., one or more senotherapeutic agents) can be determined to be an effective treatment for that mammal. When a level of one or more α-Klotho polypeptides in the first sample is less than or equal to the level of the α-Klotho polypeptide(s) in the second sample, the anti-senescence treatment (e.g., one or more senotherapeutic agents) can be determined to not be an effective treatment for that mammal.

In some cases, a first sample (e.g., a first urine sample) can be obtained from a mammal (e.g., a human) having been administered an anti-senescence treatment (e.g., having been administered one or more senotherapeutic agents), and a second sample (e.g., a second urine sample), and optionally subsequence samples, can be obtained from the mammal after the first sample was obtained. When a level of one or more α-Klotho polypeptides in the first sample is greater than the level of the α-Klotho polypeptide(s) in the second sample, the anti-senescence treatment (e.g., one or more senotherapeutic agents) can be determined to be an effective treatment for that mammal. When a level of one or more α-Klotho polypeptides in the first sample is less than or equal to the level of the α-Klotho polypeptide(s) in the second sample, the anti-senescence treatment (e.g., one or more senotherapeutic agents) can be determined to not be an effective treatment for that mammal.

In cases where two or more samples (e.g., two or more urine samples) are obtained from a mammal (e.g., a human), each sample can be obtained in any appropriate time interval. For example, a first sample and a second sample can be obtained from about 5 days to about 4 weeks apart (e.g., from about 5 days to about 4 weeks, from about 5 days to about 3 weeks, from about 5 days to about 2 weeks, from about 5 days to about 1 week, from about 1 week to about 4 weeks, from about 2 weeks to about 4 weeks, from about 3 weeks to about 4 weeks, from about 1 week to about 3 weeks, from about 1 week to about 2 weeks, or from about 2 weeks to about 3 weeks apart).

Any appropriate sample from a mammal (e.g., a human) can be assessed as described herein (e.g., for the presence, absence, or level of expression of one or more α-Klotho polypeptides). In some cases, a sample can be a biological sample. In some cases, a sample can contain one or more biological molecules (e.g., nucleic acids such as DNA and RNA, polypeptides, carbohydrates, lipids, hormones, and/or metabolites). In some cases, a sample can be a fluid sample. In some cases, a sample is not a tissue sample. Examples of samples that can be assessed as described herein include, without limitation, urine, whole blood, serum, plasma, tear, aqueous humor, and cerebrospinal Fluid (CSF). In some cases, a biological sample can be a processed sample (e.g., to isolate or extract one or more biological molecules). For example, a urine sample can be obtained from a mammal (e.g., a human) and can be assessed for the presence, absence, or level of expression of one or more α-Klotho polypeptides to determine the efficacy of the one or more senotherapeutic agents.

When assessing the efficacy of an anti-senescence treatment in a mammal (e.g., a human) as described herein (e.g., based, at least in part, a level of one or more α-Klotho polypeptides in a sample obtained from a mammal having been administered one or more senotherapeutic agents), the mammal can have been administered any one or more senotherapeutic agents. Examples of senotherapeutic agents whose efficacy in a mammal (e.g., a human) can be assessed as described herein include, without limitation, dasatinib, quercetin, navitoclax, A1331852, A1155463, fisetin, luteolin, geldanamycin, tanespimycin, alvespimycin, piperlongumine, panobinostat, FOX04-related peptides, nutlin3a, ruxolitinib, metformin, rapamycin, procyanidin C1, SSK1, Prodrug A (JHB75B), 5FURGal, Nav-Gal, PZ15227, PROTAC ARV825, and CD9-Lac/CaCO3/Rapa nanoparticles.

When assessing the efficacy of an anti-senescence treatment in a mammal (e.g., a human) as described herein (e.g., based, at least in part, a level of one or more α-Klotho polypeptides in a sample obtained from a mammal having been administered one or more senotherapeutic agents), a level of any appropriate α-Klotho polypeptide can be detected. Examples of α-Klotho polypeptides that can be used as described herein include, without limitation, an α-Klotho polypeptide having the amino acid sequence set forth in National Center for Biotechnology Information (NCBI) GenBank® or GenPept® Accession Nos. Q9UEF7, Q5VZ95, Q96KV5, Q96KW5, Q9UEI9, and Q9Y4F0.

Any appropriate method can be used to detect the presence, absence, or level of expression of one or more α-Klotho polypeptides within a sample (e.g., a sample obtained from a mammal such as a human). In some cases, the presence, absence, or level of expression of one or more α-Klotho polypeptides within a sample can be determined by detecting the presence, absence, or level of one or more α-Klotho polypeptides in the sample. For example, immunoassays (e.g., immunohistochemistry (IHC) techniques, western blotting techniques, and ELISAs) and mass spectrometry techniques (e.g., proteomics-based mass spectrometry assays or targeted quantification-based mass spectrometry assays) can be used to determine the presence, absence, or level of one or more α-Klotho polypeptides in a sample. When an immunoassay is used to determine the presence, absence, or level of one or more α-Klotho polypeptides in a sample, the immunoassay can use any appropriate antibody. Examples of antibodies that can be used in an immunoassay to determine the presence, absence, or level of one or more α-Klotho polypeptides in a sample include, without limitation, IBL America #27998 and RRID:AB_2750859. In some cases, the presence, absence, or level of expression of one or more α-Klotho polypeptides within a sample can be determined by detecting the presence, absence, or level of mRNA encoding an α-Klotho polypeptide in the sample. For example, polymerase chain reaction (PCR)-based techniques such as quantitative reverse transcription (RT)-PCR (qPCR) techniques, and RNAish can be used to determine the presence, absence, or level of mRNA encoding an α-Klotho polypeptide in the sample. In some cases, the presence, absence, or level of expression of one or more α-Klotho polypeptides within a sample can be determined by qPCR. In some cases, the presence, absence, or level of expression of one or more α-Klotho polypeptides within a sample can be determined as described in Example 1.

This document also provides methods and materials for treating a mammal (e.g., a human) having a disease or disorder characterized by a reduced level of an α-Klotho polypeptide. For example, one or more senotherapeutic agents and/or one or more inhibitors of a SASP polypeptide can be administered to a mammal (e.g., a human) to increase a level of α-Klotho polypeptides within the mammal. In some cases, increasing a level of α-Klotho polypeptides within a mammal (e.g., a human) having a disease or disorder characterized by a reduced level of an α-Klotho polypeptide can be effective to treat the mammal.

In some cases, one or more senotherapeutic agents and/or one or more inhibitors of a SASP polypeptide can be administered to a mammal (e.g., a human such as a human having a disease or disorder characterized by a reduced level of an α-Klotho polypeptide) to increase a level of an α-Klotho polypeptide within the mammal. For example, one or more senotherapeutic agents and/or one or more inhibitors of a SASP polypeptide can be administered to a mammal (e.g., a human) in need thereof (e.g., a mammal having a disease or disorder characterized by a reduced level of an α-Klotho polypeptide) to increase expression of an α-Klotho polypeptide in cells within the mammal. The term “increased level” as used herein with respect to a level of an α-Klotho polypeptide in a mammal refers to any level that is greater than the level of that α-Klotho polypeptide observed in that mammal prior to being treated as described herein (e.g., by administering one or more senotherapeutic agents). In some cases, an increased level of an α-Klotho polypeptide can be a level that is at least 5 percent (e.g., at least 10, at least 15, at least 20, at least 25, at least 35, at least 50, at least 75, at least 100, or at least 150 percent) higher than the level of that α-Klotho polypeptide prior to being treated as described herein. In some cases, when samples have an undetectable level of an α-Klotho polypeptide prior to treatment as described herein, an increased level can be any detectable level of an α-Klotho polypeptide. It will be appreciated that levels from comparable samples are used when determining whether or not a particular level is an increased level.

Any appropriate senotherapeutic agent can be administered to a mammal (e.g., a human) as described herein (e.g., to increase a level of an α-Klotho polypeptide within a mammal and/or to treat a mammal having a disease or disorder characterized by a reduced level of an α-Klotho polypeptide such as fibrosis). A senotherapeutic agent that can be used as described herein can be any type of molecule (e.g., small molecules or polypeptides). In some cases, a senotherapeutic agent can be a senolytic agent (i.e., an agent having the ability to induce cell death in senescent cells). In some cases, a senotherapeutic agent can be a senomorphic agent (i.e., an agent having the ability to suppress senescent phenotypes without cell killing). In some cases, a senotherapeutic agent can be an orally-active senotherapeutic agent. Examples of senotherapeutic agents that can be used as described herein (e.g., to increase a level of an α-Klotho polypeptide within a mammal and/or to treat a mammal having a disease or disorder characterized by a reduced level of an α-Klotho polypeptide such as fibrosis) can include, without limitation, dasatinib, quercetin, navitoclax, A1331852, A1155463, fisetin, luteolin, geldanamycin, tanespimycin, alvespimycin, piperlongumine, panobinostat, FOX04-related peptides, nutlin3a, ruxolitinib, metformin, rapamycin, procyanidin C1, SSK1, Prodrug A (JHB75B), 5FURGal, Nav-Gal, PZ15227, PROTAC ARV825, and CD9-Lac/CaCO3/Rapa nanoparticles. In some cases, a senotherapeutic agent can be as described elsewhere (see, e.g., Kirkland et al., Exp. Gerontol., 68:19-25 (2015)).

Any appropriate inhibitor of a SASP polypeptide can be administered to a mammal (e.g., a human) as described herein (e.g., to increase a level of an α-Klotho polypeptide within a mammal and/or to treat a mammal having a disease or disorder characterized by a reduced level of an α-Klotho polypeptide such as fibrosis). An inhibitor of a SASP polypeptide can inhibit any SASP polypeptide. Examples of SASP polypeptides that can be used as described herein (e.g., to increase a level of an α-Klotho polypeptide within a mammal and/or to treat a mammal having a disease or disorder characterized by a reduced level of an α-Klotho polypeptide such as fibrosis) can include, without limitation, activin A polypeptides and interleukin 1α (IL-1α) polypeptides. An inhibitor of a SASP polypeptide can inhibit SASP polypeptide activity or SASP polypeptide expression. Examples of compounds that can reduce or eliminate polypeptide activity of a SASP polypeptide include, without limitation, antibodies (e.g., neutralizing antibodies) and small molecules that target (e.g., target and bind) to a SASP polypeptide. When a compound that can reduce or eliminate polypeptide activity of a SASP polypeptide is a small molecule that targets (e.g., targets and binds) to a SASP polypeptide, the small molecule can be in the form of a salt (e.g., a pharmaceutically acceptable salt). Examples of compounds that can reduce or eliminate polypeptide expression of a SASP polypeptide include, without limitation, nucleic acid molecules designed to induce RNA interference of polypeptide expression of a lipase (e.g., a siRNA molecule or a shRNA molecule), antisense molecules, and miRNAs. In some cases, an inhibitor of a SASP polypeptide can be as described in Example 1.

When one or more senotherapeutic agents are administered to a mammal (e.g., a human) having a disease or disorder characterized by a reduced level of an α-Klotho polypeptide, the mammal can have any disease or disorder characterized by a reduced level of an α-Klotho polypeptide. In some cases, a reduced level of an α-Klotho polypeptide can refer to a urinary level that is less than 293.49±115.48 ng of α-Klotho polypeptides per mg creatinine in a human. Examples of diseases and disorders that are characterized by a reduced level of an α-Klotho polypeptide and can be treated as described herein (e.g., by administering one or more senotherapeutic agents) include, without limitation, fibrosis (e.g., idiopathic pulmonary fibrosis (IPF)), chronic kidney disease, acute kidney injury, diabetes, cancer, dementia, Alzheimer's disease, and arteriosclerosis.

When one or more senotherapeutic agents are administered to a mammal (e.g., a human) having fibrosis (e.g., IPF), the one or more senotherapeutic agents can be effective to 10 reduce or eliminate one or more (e.g., one, two, three, four, five or more) symptoms of the fibrosis. Examples of symptoms of fibrosis that can be reduced or eliminated as described herein include, without limitation, shortness of breath (dyspnea), a dry cough, fatigue, unexplained weight loss, aching muscles and joints, chest pain, and leg swelling. For example, one or more senotherapeutic agents and/or one or more inhibitors of a SASP polypeptide can be administered to a mammal (e.g., a human) in need thereof (e.g., a mammal having fibrosis such as IPF) as described herein to reduce one or more symptoms of fibrosis in the mammal by, for example, 10, 20, 30, 40, 50, 60, 70, 80, 90, 95, or more percent.

In some cases, methods for treating a mammal (e.g., a human) as described herein (e.g., by administering one or more senotherapeutic agents to increase a level of one or more α-Klotho polypeptides to, for example, treat a disease or disorder characterized by a reduced level of an α-Klotho polypeptide such as fibrosis (e.g., IPF)) can include administering to the mammal one or more (e.g., one, two, three, four, or more) senotherapeutic agents as the sole active ingredient to increase a level of one or more α-Klotho polypeptides in the mammal. For example, a composition containing one or more senotherapeutic agents can include the one or more senotherapeutic agents as the sole active ingredient in the composition that is effective to increase a level of one or more α-Klotho polypeptides in a mammal. For example, a composition containing one or more senotherapeutic agents can include the one or more senotherapeutic agents as the sole active ingredient in the composition that is effective to treat a mammal having a disease or disorder characterized by a reduced level of an α-Klotho polypeptide (e.g., fibrosis such as IPF).

In some cases, methods for treating a mammal (e.g., a human) as described herein (e.g., by administering one or more senotherapeutic agents to increase a level of one or more α-Klotho polypeptides to, for example, treat a disease or disorder characterized by a reduced level of an α-Klotho polypeptide such as fibrosis (e.g., IPF)) also can include administering to the mammal one or more (e.g., one, two, three, four, five or more) additional agents used to increase α-Klotho polypeptide expression and/or to treat fibrosis (e.g., IPF) to the mammal and/or performing therapies used to treat fibrosis (e.g., IPF) on the mammal. For example, a combination therapy used to increase α-Klotho polypeptide expression and/or to treat fibrosis (e.g., IPF) can include administering to the mammal (e.g., a human) one or more senotherapeutic agents described herein and one or more (e.g., one, two, three, four, five or more) agents used to increase α-Klotho polypeptide expression and/or to treat fibrosis (e.g., IPF). Examples of agents that can be administered to a mammal to increase α-Klotho polypeptide expression and/or to treat fibrosis (e.g., IPF) include, without limitation, PPARγ agonists, losartan, statin HMG-CoA reductase inhibitors, vitamin D derivatives, and any combinations thereof. In some cases, an agent that can increase α-Klotho polypeptide expression can be as described elsewhere (see, e.g., Zhang et al., Kidney Int., 74(6):732-9 (2008); Lim et al., J. Renin. Angiotensin Aldosterone Syst., 15(4):487-90 (2014); Kuwahara et al., Int. J. Cardiol., 123(2):84-90 (2008); and Hajialilo et al., Rheumatol. Int., 37(10):1651-7 (2017)). In cases where one or more senotherapeutic agents are used in combination with additional agents used to increase α-Klotho polypeptide expression and/or to treat fibrosis (e.g., IPF), the one or more additional agents can be administered at the same time (e.g., in a single composition containing both one or more senotherapeutic agents and the one or more additional agents) or independently. For example, one or more senotherapeutic agents described herein can be administered first, and the one or more additional agents administered second, or vice versa.

In some cases, a combination therapy used to increase α-Klotho polypeptide expression and/or to treat fibrosis (e.g., IPF) can include administering to the mammal (e.g., a human) one or more (e.g., one, two, three, four, or more) senotherapeutic agents described herein and performing one or more (e.g., one, two, three, four, five or more) additional therapies used to treat fibrosis (e.g., IPF) on the mammal. Examples of therapies used to treat fibrosis (e.g., IPF) include, without limitation, oxygen therapy, pulmonary rehabilitation, and/or lung transplantation. In cases where one or more senotherapeutic agents described herein are used in combination with one or more additional therapies used to treat fibrosis (e.g., IPF), the one or more additional therapies can be performed at the same time or independently of the administration of one or more senotherapeutic agents described herein. For example, one or more senotherapeutic agents described herein can be administered before, during, or after the one or more additional therapies are performed.

In certain instances, a course of treatment and the severity of one or more symptoms related to a condition being treated (e.g., fibrosis such as IPF) can be monitored. Any appropriate method can be used to determine whether or not the severity of a symptom is reduced. For example, the presence, absence, or level of expression of one or more α-Klotho polypeptides within a sample (e.g., a urine sample) obtained from a mammal (e.g., a human) being treated for fibrosis (e.g., IPF) can be used to monitor a course treatment as described herein.

The invention will be further described in the following examples, which do not limit the scope of the invention described in the claims.

### EXAMPLES

### Example 1: Orally-Active, Clinically-Translatable Senolytics Restore α-Klotho in Mice and Humans

This Example demonstrates that α-Klotho and cellular senescence are inversely related. For example, orally-active small molecule senolytic agents can be used to increase expression of an α-Klotho polypeptide. In addition, the level of an α-Klotho polypeptide in a sample can be used as a biomarker to determine senescent cell burden and/or the efficacy of one or more senolytic agents.

### Methods

### Study Design Overview

A translational study of α-Klotho was conducted by first establishing α-Klotho signalling mechanisms in cultured human primary senescent cells. Then the casual links between α-Klotho and senescent cells were explored in vivo following genetic clearance of senescent cells and senolytic drug administration in naturally aged, DIO, and senescent cell-transplanted mice administered senolytic drugs vs. vehicle. Finally, urinary α-Klotho was measured before and after senolytic drug treatment in a post hoc analysis of a single-arm, open-label trial in older adults with IPF (Justice et al., EBioMedicine, 40:554-63 (2019)).

### Reagents

Human primary cells were obtained from commercial sources, passed quality control procedures, and were certified by the commercial sources. All reagents were validated by the manufacturer and/or has been previously cited in the literature. Detailed information about these reagents is provided in Table. 1.

TABLE 1
Reagents used in this study.

| Reagent names | Cat# | Company |
|---|---|---|
| anti-Interleukin 1β | 508202 | Biolegend |
| Anti-Interleukin 18 | PA5-47803 | Thermo Fisher Scientific |
| Anti- CXCL1 | MAB275 | R&D Systems |
| Anti-Interleukin 6 | MAB2061 | R&D Systems |
| Anti-Activin A | MAB3381 | R&D Systems |
| Anti-TNF-α | 7321S | Cell Signaling Technology |
| Anti-PAI1 | MAB1786 | R&D Systems |
| Anti-α-Klotho antibody | MA5-32784 | Invitrogen |
| Recombinant human IL-1α protein | 00-LA-010 | R&D Systems |
| Recombinant human activin A | 338-AC | R&D Systems |
| Mouse TBP primers | Mm00446971_m1 | Invitrogen |
| Mouse IL-1α primers | Mm00439620_m1 | Invitrogen |
| Mouse Activin A primers | Mm00434339_m1 | Invitrogen |
| Mouse α-Klotho primers | Mm00434339_m1 | Invitrogen |
| Human TBP primers | Hs00427620_m1 | Invitrogen |
| Human CDKN2A primers | Hs00923894_m1 | Invitrogen |
| Human CDKN1A primers | Hs00355782_m1 | Invitrogen |
| Human TNFα primers | Hs00174128_m1 | Invitrogen |
| Human IL6 primers | Hs00174131_m1 | Invitrogen |
| Human CXCL8 primers | Hs00174103_m1 | Invitrogen |
| Human IL18 primers | Hs01555410_m1 | Invitrogen |
| Human PAI-1 primers | Hs01126607_g1 | Invitrogen |
| Human IL-1α primers | Hs00174092_m1 | Invitrogen |
| Human IL-1β primers | Hs01555410_m1 | Invitrogen |
| Human CXCL1 primers | Hs00236937_m1 | Invitrogen |
| Human Activin A primers | Hs01081598_m1 | Invitrogen |
| Human Renal Glomerular Endothelial cells | 4000 | ScienCell Research Laboratories |
| (HKEs) | | |
| Human Umbilical Vein Endothelial Cells | C2519A | Lonza |
| (HUVECs) | | |
| Human Astrocytes | 1800 | ScienCell Research Laboratories |
| (HBAs) | | |

### Animals

Wild-type C57BL/6 mice (young=8-month-old, male) were purchased from Charles River. INK-ATTAC mice were as described elsewhere (Baker et al., Nature, 479(7372):232-6 (2011)). Briefly, in INK-ATTAC mice, the ATTAC gene is driven by a senescence-related p16^{Ink4a} promoter fragment. The fusion protein product of the ATTAC “suicide” gene contains a mutated FKBP moiety that can be cross-linked by AP20187, thereby activating the ATTAC protein caspase-8 moieties, causing apoptosis. INK-ATTAC mice were bred, genotyped, and aged in a pathogen-free and maintained at 23-24° C. under a 12 hours light, 12 hours dark regimen. Mice had free access to water and a 20% protein by weight, 5% fat (13.2% fat by calories), and 6% fiber diet (Lab Diet). DIO mice were fed a diet in which 60% of calories were from fat (Research Diets) for 7-8 months before experiments.

### Human IPF Trial

This was a post hoc analysis of a study described elsewhere (Justice et al., EBioMedicine, 40:554-63 (2019)). Briefly, this study was conducted at the Clinical Research Units of WFSM (n=2) and UTHSCSA (n=18). Intermittent D (100 mg/day) plus Q (1,250 mg/day) were orally administered over three consecutive days for three weeks (i.e. 9 total participant administered dosing days) at the two outpatient clinical research centers using a single-arm, open-label design.

### Cell Transplantation

Wild-type C57BL/6 mice were randomly assigned to be transplanted with senescent (induced by 10Gy x-ray) or non-senescent adipocyte progenitors or vehicle (phosphate buffered saline (PBS)) and matched for body weight across groups. Mouse preadipocytes were isolated and cultured. Mice were anesthetized using isoflurane and 2 million cells were transplanted intraperitoneally (i.p.) in 150 μL PBS through a 22G needle.

Drug treatments INK-ATTAC mice were randomly assigned to AP20187 or vehicle groups. AP20187 was purchased from Clontech. Vehicle (10% ethanol, 30% polyethylene glycol, 60% Phosal) or AP20187 in vehicle was injected i.p. (10 mg/kg) for 3 consecutive days every 2 weeks for 4 weeks. Wild-type C57BL/6 mice were randomly assigned to D+Q, Fisetin (F), or vehicle treatments. Treatments were started at age 26-27 months for old animals, and for DIO mice after 7-8 months of high fat-feeding beginning at 4 months of age. Mice were treated every 20 days with D+Q, F, or vehicle by oral gavage for 3 consecutive days in each of 3 cycles over 2 months (9 doses in total).

### Cell Culture

HKEs (ScienCell Research Laboratories, #4000), HUVECs (Lonza, #C2519A), and HBAs (ScienCell Research Laboratories, #1800) were cultured following the suppliers' directions. All cells purchased from commercial sources were validated by the manufacturers (Table 1). Briefly, conditioned medium (CM) from senescent or non-senescent HKEs, HUVECs, or HBAs was filtered (0.2 μm) before being added to target non-senescent HKEs, HUVECs, or HBAs, respectively, with or without neutralizing antibodies against Interleukin-1β (IL-1β; Biolegend, #508202, RRID:AB_315514), Interleukin-18 (IL-18; Thermo Fisher Scientific, #PA5-47803, RRID:AB_2606212), CXCL1 (R&D Systems, #MAB275, RRID:AB_2292460), Interleukin-6 (IL-6; R&D Systems, #MAB2061, RRID:AB_354281), activin A (R&D Systems, #MAB3381), Tumour Necrosis Factor α (TNF-α) (Cell Signaling Technology, #7321S), or Plasminogen Activator Inhibitor-1 (PAI-1; R&D Systems, #MAB1786) for 48 hours. Cells were then collected for assay by qPCR.

Non-senescent HKEs, HUVECs, and HBAs were co-cultured with recombinant human IL-1α protein (R&D Systems, #00-LA-010) or recombinant human activin A (R&D Systems, #338-AC) for 48 hours, when cells were assayed by qPCR.

qPCR

Each cDNA sample was generated by reverse transcription using 1 μg RNA following the manufacturer's protocol (Thermo Fisher Scientific). Reverse transcription involved incubation for 10 minutes at 25° C., 120 minutes at 37° C., 5 minutes at 85° C., and holding at 4° C. using Taqman Fast Advanced Master Mix (Thermo Fisher Scientific). TBP was used as a control. Data were analysed by the ΔΔCt method.

### Human and Mouse Urinary α-Klotho

α-Klotho was assayed by ELISA in 1:100 diluted mouse urine (IBL America, #27601). Human urine α-Klotho was assayed by ELISA (IBL America, #27998, RRID:AB_2750859). Urine α-Klotho measurements are expressed as a function of urine volume, creatinine (R&D Systems, #KGE005), and/or cystatin C (R&D Systems, #MSCTC0).

### Senescent Cell Quantification by Mass Cytometry (CyTOF)

CyTOF experiments were performed as described elsewhere (Palmer et al., Aging Cell., 18(3):e12950 (2019)).

### Immunofluorescent Staining

Mouse brains were processed for paraffin embedding, cut into 4 μm-thick sagittal sections, deparaffinized in Histoclear (National Diagnostics), and rehydrated in decreasing percentages of ethanol diluted in water. Prior to staining, antigen retrieval was performed by incubating sections in Tris EDTA pH9 buffer in a steamer for 20 minutes and cooling to room temperature. Sections were washed with PBS and blocked with 5% Normal Goat Serum and 0.3% Tween20 in PBS-BSA 0.1% for 30 minutes. Rabbit anti-mouse α-Klotho antibody (Invitrogen, #MA5-32784) was diluted 1:50 in blocking solution, added to sections, and incubated overnight at 4° C. Slides were washed with PBS and incubated with secondary goat anti-rabbit 647 antibody (Invitrogen, A-21244, RRID: AB_2535812) for 1 hour at room temperature in the dark. Slides were washed and mounted in ProLong Gold Antifade mountant with DAPI (Invitrogen, P36935).

### Imaging and Analysis

Stained α-Klotho mouse whole brain sections were scanned using an Axio Scanner Z1 (Zeiss) at 20× magnification. Photoshop CC 19.1 (Adobe, Inc.) was used to virtually dissect the choroid plexus, whole cerebellum, and background samples, avoiding major blood vessels with erythrocytes or tissue folding that could interfere with fluorescence intensity calculations. Using ImageJ FIJI, the region of interest (ROI) was selected for analysis to exclude blood vessels and folding of tissue that can interfere with intensity measurements. ROI areas were quantified for raw intensity and then corrected with background on the same section. Results are shown as the corrected mean intensity, calculated as corrected intensity divided by area.

### Western Blotting

Tissue extracts were homogenized using a Bead Mill 24 homogenizer (Waltham) and lysed in NETN buffer (20 mM Tris-HCl, pH 8.0, 100 mM NaCl, 1 mM EDTA, 0.5% Nonidet P-40), 5 mM NaF, a protease inhibitor cocktail (Millipore Sigma), and 5 mM nicotinamide. After incubating 30 minutes at 4° C., samples were centrifuged at 15,000 rpm for 10 minutes at 4° C. Protein concentrations of the supernatants were determined by the Bradford protein assay (BioRad). Lysates were separated by SDS-PAGE and transferred by electrophoresis to PVDF membranes (Millipore Sigma). Membranes were immunoblotted with primary mouse α-Klotho antibody (R&D Systems, AF1819) at 1:1000 dilution and GAPDH antibody (Cell Signaling Technology, 97166) at 1:1000 dilution. Enhanced chemiluminescence detection was performed using SuperSignal West Pico or Femto Chemiluminescence Substrate (Thermo Scientific). All blots were imaged with a GelDoc Go Imaging System (BioRad).

### Statistical Analyses

At least four independent replicates were studied in all cell culture experiments. Randomisation: Mice were randomized using a random number generator in all experiments. Blinding: Experiments were conducted with the investigators being blinded as to treatment, group allocation for immunofluorescence (IF) staining, ELISA assays, quantitative PCR (qPCR), and quantification of protein expression. Sample size determination: Sample sizes were chosen based on the means and variation of preliminary data to achieve at least 80% power and allow for a 5% type I error. Inclusion exclusion: The animals were matched for age, gender, and body weight. No animals or experimental data were excluded. Inclusion and exclusion criteria for the clinical trial from which de-identified urine was acquired for post hoc analysis were as described elsewhere (Justice et al., EBioMedicine, 40:554-63 (2019)).

All data were plotted and analyzed for statistical significance using Prism 9.0 (GraphPad) for cell culture and animal experiments. R² (same as η²), calculated in Prism, was reported as the effect size. Comparisons between a single treated group and vehicle were made using non-paired T-tests. When comparisons were made between multiple treatment levels, a mixed effects repeated measures model was used, accounting for the repeated measures within each subject. For IPF clinical trial data, the following statistical methods were used and due to the wide range of the values, a log transformation was performed before analysis. Zeros (n=3) were replaced with half of the minimum non-zero value before log transformation. Imputation using half of the minimum was used to impute zeros due to under-detection. This imputation method is available in MetaboAnalyst 2.0, a web server for data analysis and interpretation. MetaboAnalyst 2.0 allows selected analyses to avoid divide-by-zero problems to process missing values, which can be replaced by the half of the minimum value found in the dataset by default. Wilcoxon signed rank tests (two-sided) were used to test differences before and after treatment in human trial data since the data were not normally distributed. Both Spearman's rho and Kendall's tau are non-parametric tests for association and test the same null hypothesis and both control for type I error at the nominal level. In this study, Spearman's rank correlation was used to test the association between urinary α-Klotho and SASP factors in urine.

### Results

### Senescent Cells Decrease α-Klotho Through Paracrine Mechanisms

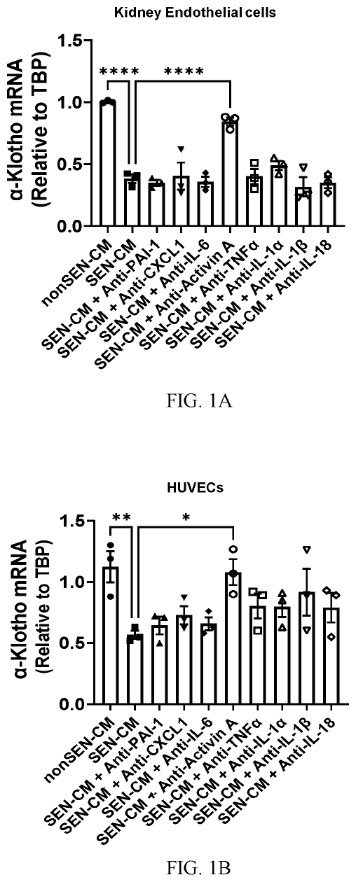

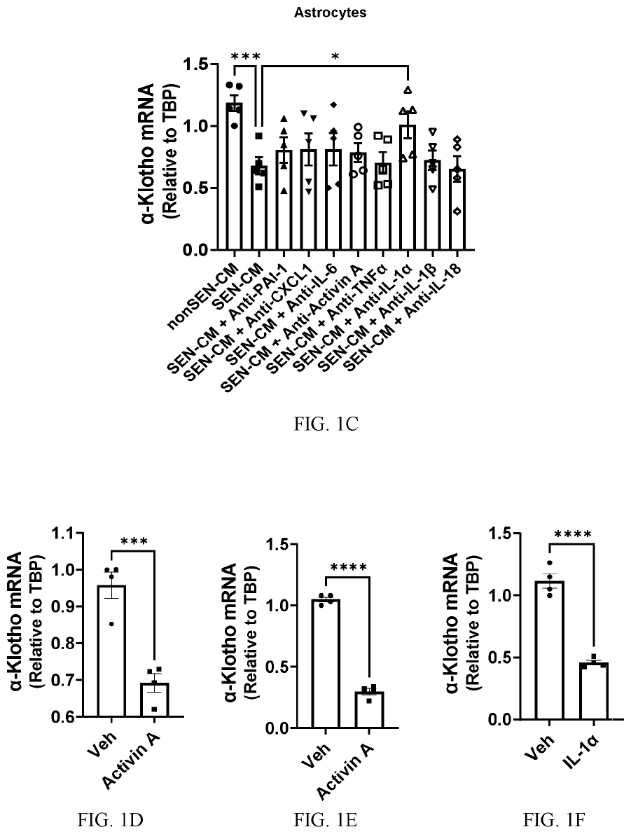

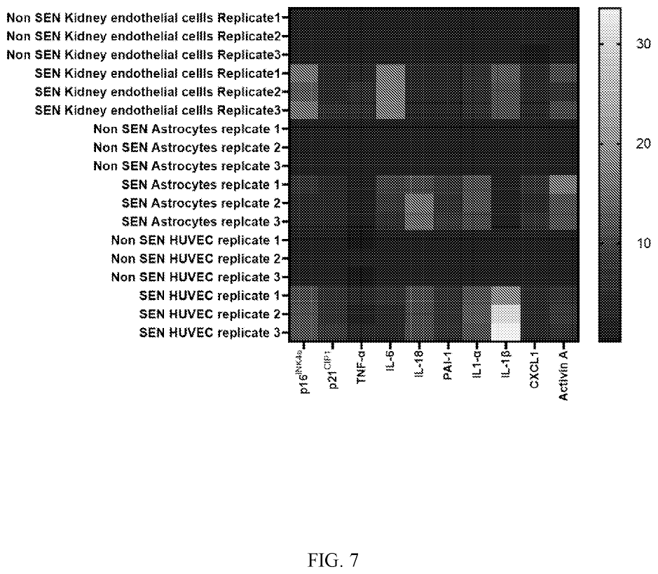

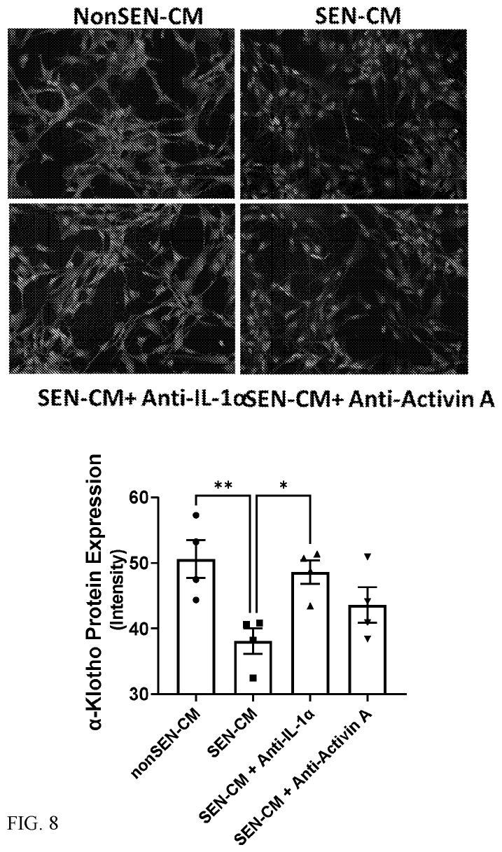

HKEs, HUVECs, and HBAs were radiated (20Gy x-ray). By 25 days after radiation, the cells had become senescent (FIG. 7). To determine if senescent cells can directly impact α-Klotho expression, HKEs, HUVECs, and HBA were exposed to conditioned medium (CM) from cultured senescent or non-senescent human HKEs, HUVECs, or HBAs, respectively. Senescent cell CM exposure resulted in decreased α-Klotho expression in the non-senescent cells (FIG. 1A-C, FIG. 8). These reductions were attenuated in non-senescent HKEs or HUVECs by neutralizing antibodies against activin A, and in non-senescent HBAs by anti-IL-1α (FIGS. 1A-C). Conversely, treating human non-senescent HKEs, HUVECs, or HBAs with recombinant activin A or IL-1α caused decreased expression of α-Klotho (FIG. 1D). Thus, components of the SASP can decrease α-Klotho in non-senescent cells.

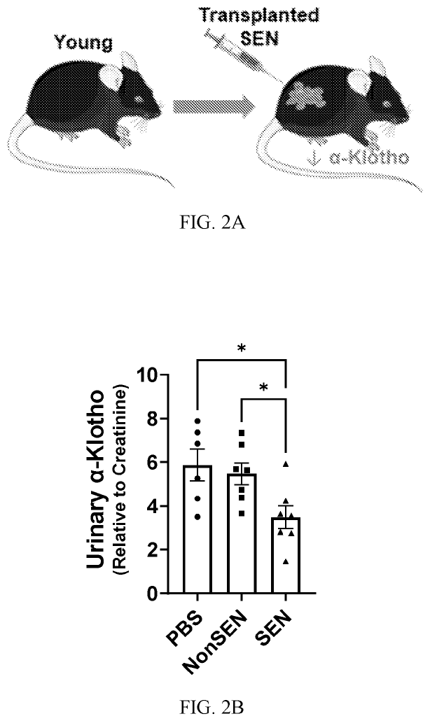

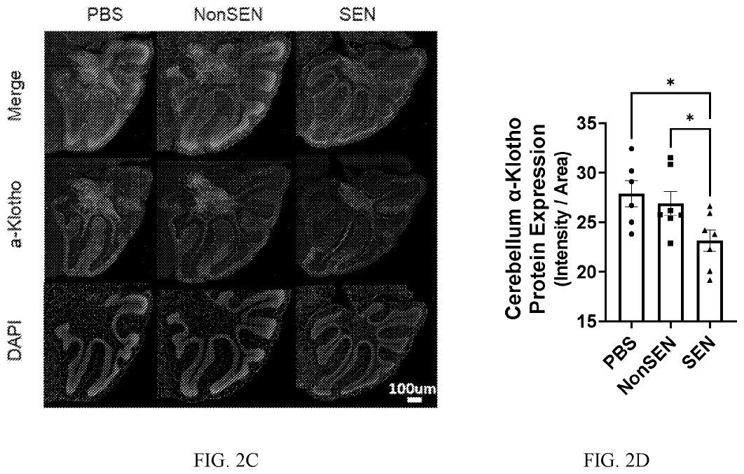

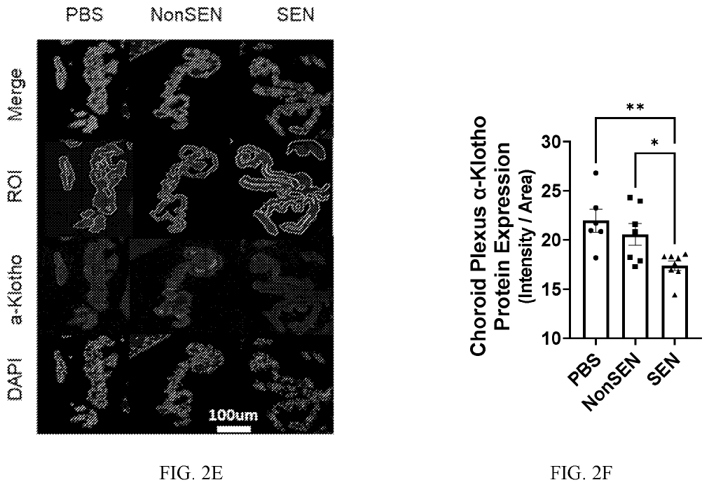

To test causality further, it was ascertained if senescent cells can decrease α-Klotho in vivo by transplanting small numbers of senescent or non-senescent adipocyte progenitors vs. vehicle (PBS) i.p. into young mice (8-month-old) (FIG. 2A). Transplanting these mice with senescent cells decreased urinary, cerebellar, and choroid plexus α-Klotho protein (FIGS. 2B-F) compared to control non-senescent cell transplanted- or PBS-treated mice.

Genetic Clearance of p16^{Ink4a}-Expressing Senescent Cells Increases α-Klotho

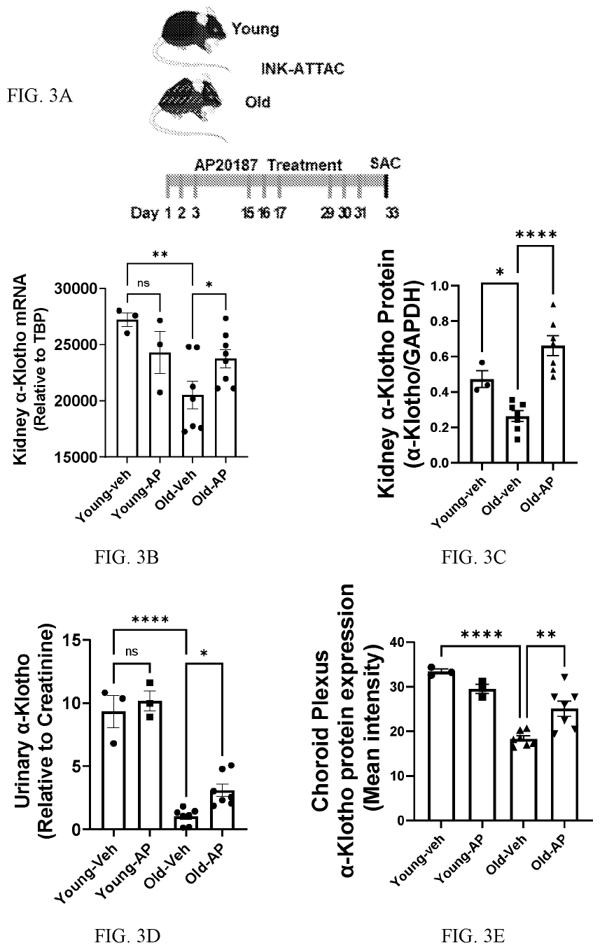

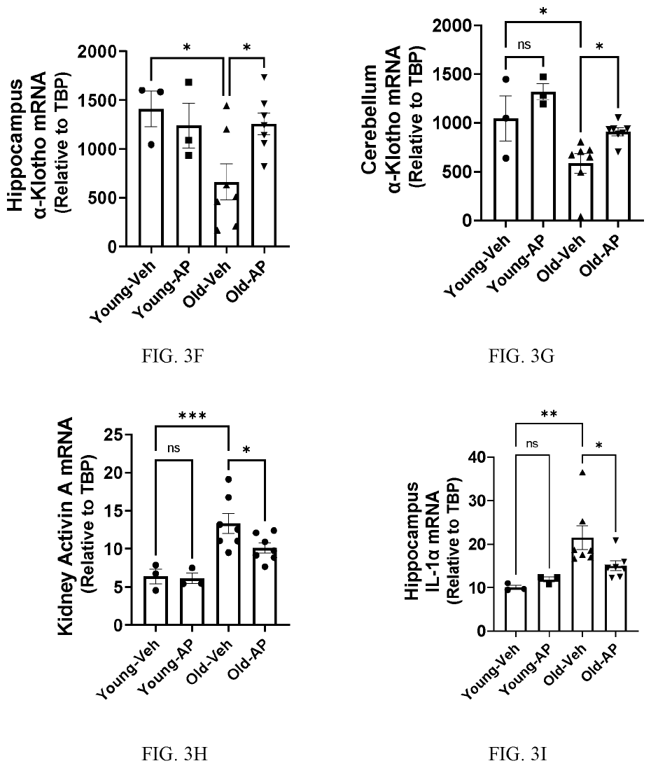

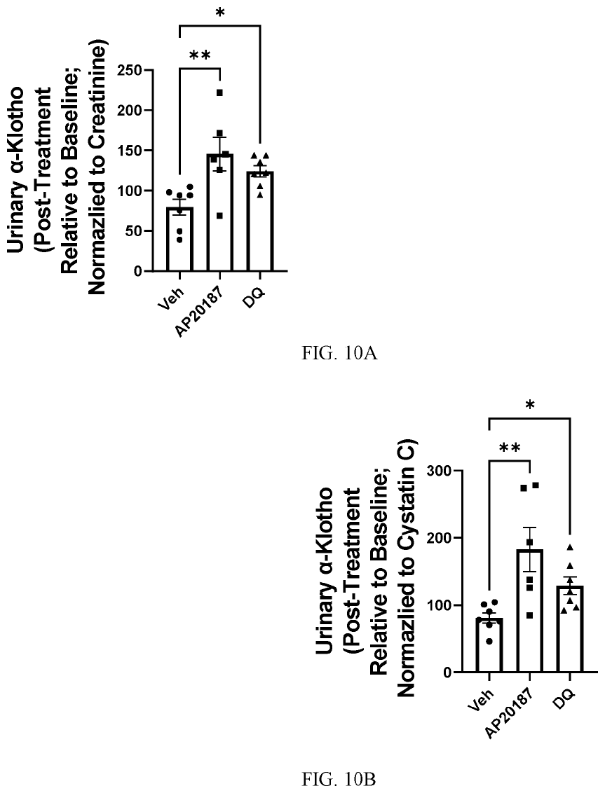

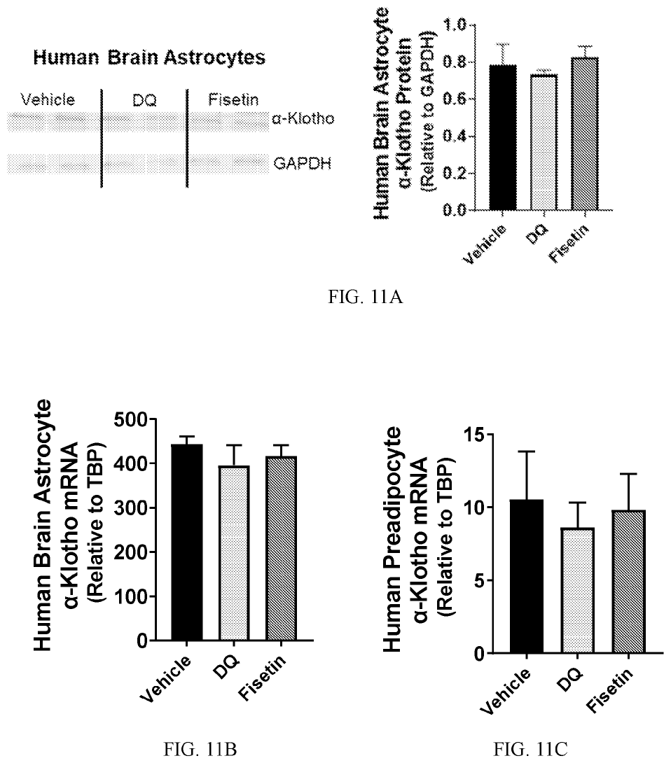

Next, it was determined if removal of p16^{Ink4a}-expressing cells, many of which are senescent, increases α-Klotho in old mice. To accomplish this, transgenic INK-ATTAC mice were used in which the drug AP20187 dimerizes the ATTAC “suicide” caspase-8 moiety-containing fusion protein, causing death of cells that highly express p16^{Ink4a}. Conditions alleviated by decreasing p16^{Ink4a+} cells in INK-ATTAC mice parallel those alleviated by increasing α-Klotho, including age- or high fat diet-induced adipose tissue and metabolic dysfunction, age-related osteoporosis, and bleomycin-induced lung fibrosis. Eight- and 26-27-month-old INK-ATTAC mice were administrated 10 mg/kg AP20187 in cycles of 3 consecutive days every 15 days and euthanized 5 days after the third 3-day course (9 doses in total) of AP20187 (FIG. 3A). Kidney, urinary, and brain α-Klotho, which was lower in old mice than it was in younger INK-ATTAC mice, was increased by AP20187 in the old mice (FIGS. 3B-D, E-G, FIG. 10). Consistent with results in the in vitro experiment (FIGS. 1A-F), activin A and IL-1α were high in kidneys and brains of old INK-ATTAC mice, respectively, but decreased after AP20187 treatment (FIGS. 3H&I), suggesting that senescent cells contribute to the age-related decline in α-Klotho in part through the SASP factors, activin A and IL-1α.

### Senolytics Increase α-Klotho In Vivo

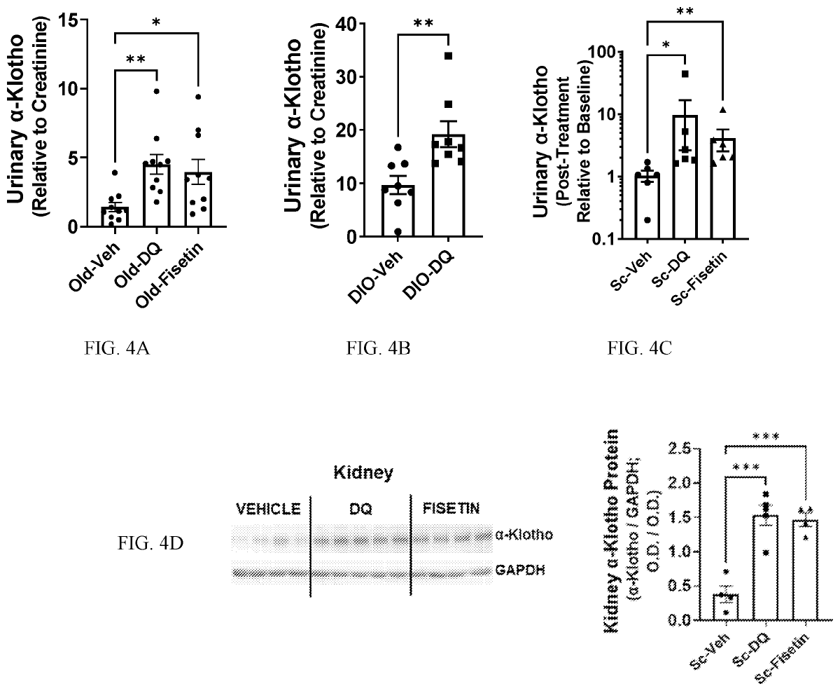

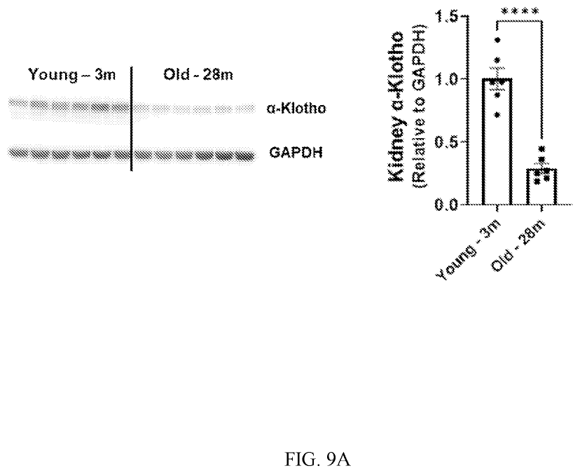

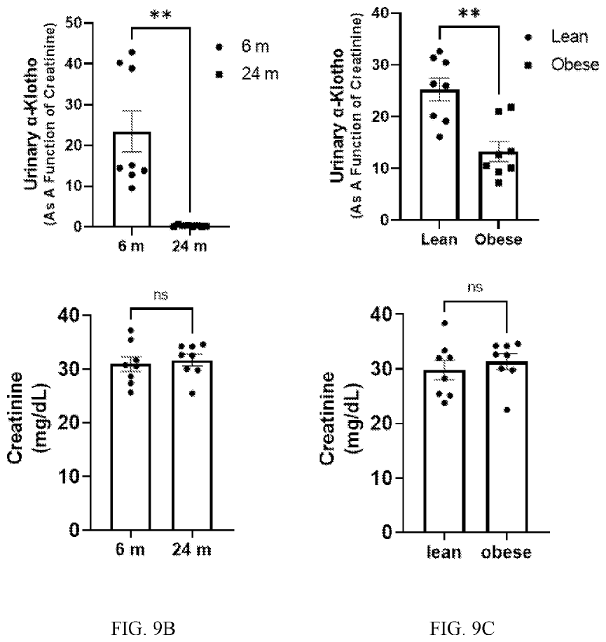

Treating mice orally with the senolytics, D+Q or F, increased urine α-Klotho in aged mice (FIG. 4A). In young wild-type DIO mice, which have an increased burden of senescent cells compared to lean mice and a decline in urinary α-Klotho (FIG. 9), D+Q was effective in increasing urinary α-Klotho (FIG. 4B, FIG. 410). Both D+Q and F increased urinary α-Klotho in young mice that had been transplanted with senescent cells (FIG. 4C). Kidney α-Klotho, which was lower in old mice than it was in young mice (FIG. 9), was increased by D+Q in the old mice (FIG. 4D).

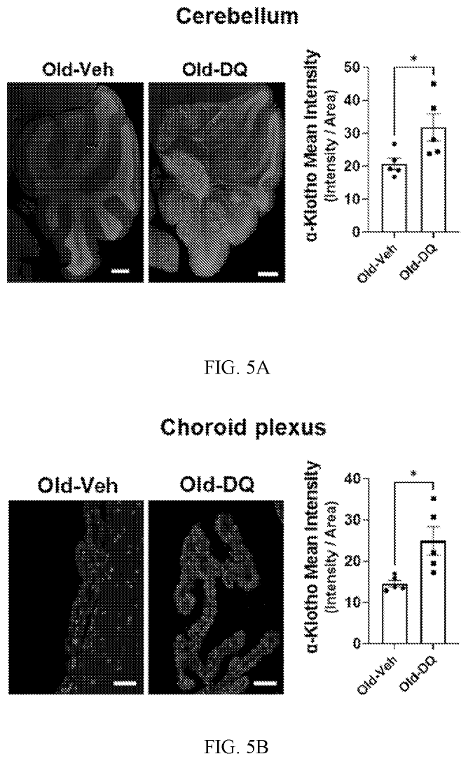

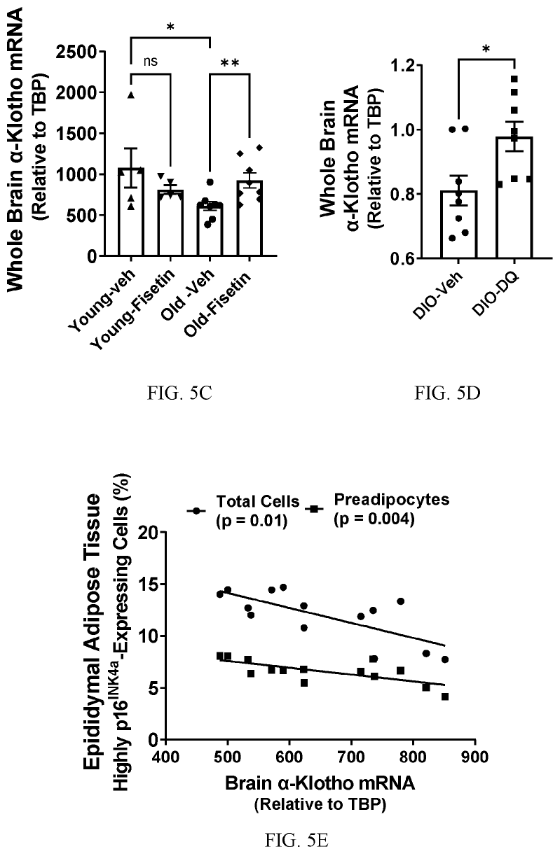

The brain is another primary site of α-Klotho production. It was found that senolytics increase α-Klotho protein in the cerebellum and choroid plexus (FIGS. 5A-B) as well as α-Klotho mRNA in whole brains of old mice in which α-Klotho decreases with ageing (FIG. 5C). In young, obese mice, senolytics increased brain α-Klotho (FIG. 5D). Furthermore, α-Klotho was inversely related to adipose tissue senescent cell burden in untreated obese mice (FIG. 5E).

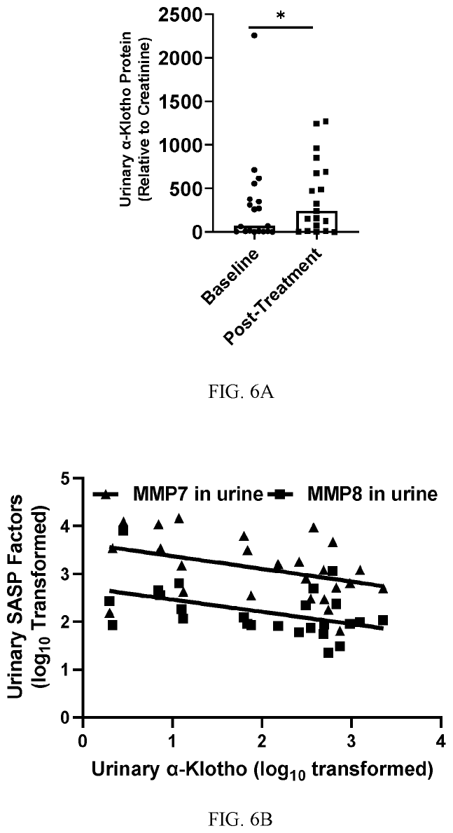

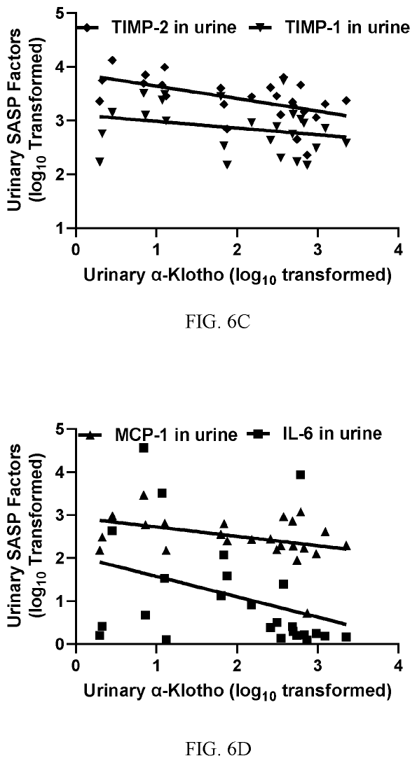

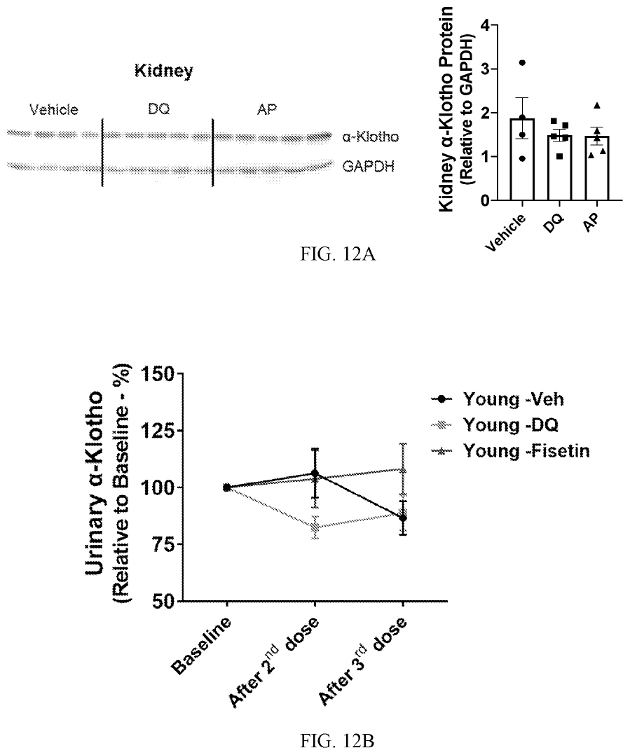

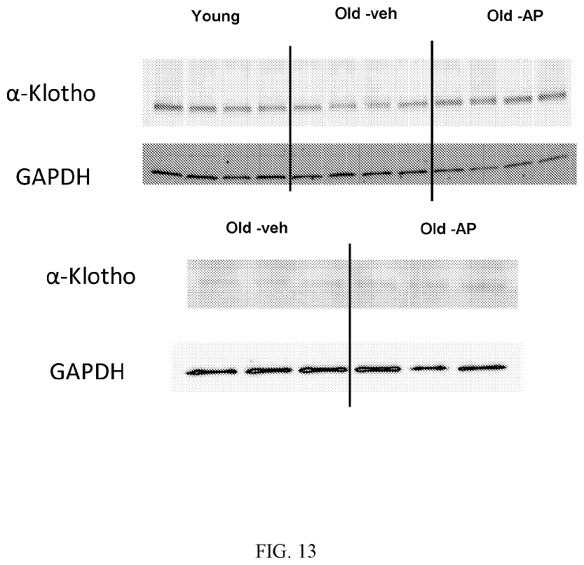

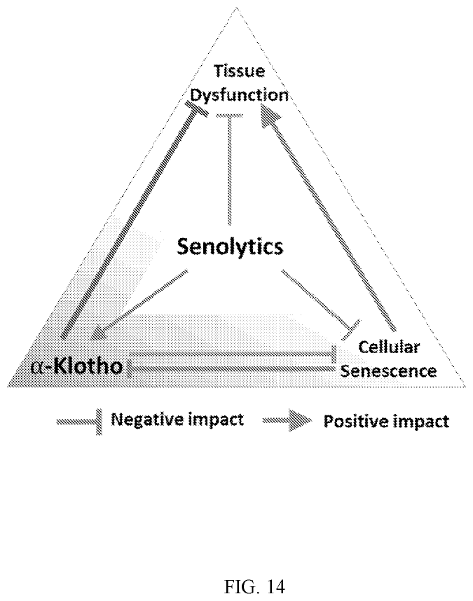

To determine if senolytics increase α-Klotho through direct, off-target effects in α-Klotho-expressing non-senescent cells, human cultured non-senescent preadipocytes or astrocytes were exposed to D+Q or F (FIG. 11). Treatment with senolytics did not increase α-Klotho in these cultured non-senescent cells. Treating young INK-ATTAC mice with AP20187 or D+Q also did not increase kidney α-Klotho (FIG. 12A). Furthermore, senolytics did not increase α-Klotho in urine of young mice (FIG. 6B). Thus, senescent cell targeting strategies do not appear to increase α-Klotho when senescent cell burden is low, consistent with increases in α-Klotho being due to removal of senescent cells, rather than other mechanisms.

### Senolytics Increase α-Klotho in Humans

In patients who have IPF, urinary α-Klotho was increased after (392.79±95.24, normalized to urinary creatinine), compared to before (293.49±115.48, normalized to urinary creatinine), D+Q administration (FIG. 6A). It was also found that urinary SASP factors were inversely correlated with urinary α-Klotho (FIGS. 6B-D, Table 2).

TABLE 2
Urinary α-Klotho in humans with IPF correlates with urinary SASP factors.

| Spearman's rank | Urinary α-Klotho vs. Urinary SASP | | | | | |
|---|---|---|---|---|---|---|
| Correlation Test | IL-6 | MCP-1 | MMP-7 | MMP-8 | TIMP-1 | TIMP-2 |
| FDR-corrected r | −0.558 | −0.353 | −0.352 | −0.407 | −0.305 | −0.602 |
| value | | | | | | |
| FDR-corrected p | 0.00035 | 0.0289 | 0.0289 | 0.0157 | 0.055 | 0.0002 |
| value | | | | | | |

Subjects with IPF were administered 3 courses of D+Q, each course being 3 sequential days of administration, for a total of 9 doses over 3 weeks. Urinary α-Klotho and SASP factors were assayed at baseline and 5 days after the last D+Q treatment as a function of creatinine. Spearman correlation tests were used for analyzing correlations.

Together, these results demonstrate that a level of one or more α-Klotho polypeptides in a sample (e.g., a urine sample) obtained from a mammal (e.g., a human) having been administered one or more senotherapeutic agents can be used to determine the efficacy of the one or more senotherapeutic agents. These results also demonstrate that one or more senotherapeutic agents and/or one or more inhibitors of a SASP polypeptide can be administered to a mammal (e.g., a human) to increase a level of α-Klotho polypeptides within the mammal.

### Example 2: Assessing the Efficacy of an Anti-Senescence Treatment

A first urine sample is obtained from a human prior to the human being administered an anti-senescence treatment (e.g., prior to being administered one or more senotherapeutic agents), and a second urine sample is obtained from the human after the human has been administered the anti-senescence treatment.

The first and second urine samples are assayed to determine the presence, absence, or level of one or more α-Klotho polypeptides in the samples.

When a level of one or more α-Klotho polypeptides in the first urine sample is greater than the level of the α-Klotho polypeptide(s) in the second urine sample, the anti-senescence treatment (e.g., one or more senotherapeutic agents) can be determined to be an effective treatment for that human.

When a level of one or more α-Klotho polypeptides in the first urine sample is less than equal to the level of the α-Klotho polypeptide(s) in the second urine sample, the anti-senescence treatment (e.g., one or more senotherapeutic agents) can be determined to not be an effective treatment for that human.

### Example 3: Increasing α-Klotho Polypeptide Expression

A human in need thereof (e.g., a human having a disease or disorder associated with reduced α-Klotho polypeptides) is administered or self-administers one or more orally-active senotherapeutic agents (e.g., dasatinib and/or quercetin). The administered senotherapeutic agent(s) can increase a level of one or more α-Klotho polypeptides in the human (e.g., can increase α-Klotho polypeptide expression by cells within the human).

### Example 4: Treating IPF

A human identified as having IPF is administered or self-administers one or more orally-active senotherapeutic agents (e.g., dasatinib and/or quercetin). The administered senotherapeutic agent(s) can reduce the severity of one or more symptoms of IPF.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for assessing efficacy of an anti-senescence treatment, wherein said method comprises:
(a) detecting a level of an α-Klotho polypeptide in a first urine sample obtained from a mammal prior to or within 24 hours of administration of said anti-senescence treatment to said mammal;
(b) detecting a level of said α-Klotho polypeptide in a second urine sample obtained from said mammal at least 120 hours after administration of said anti-senescence treatment to said mammal;
(c) identifying said anti-senescence treatment as being effective if the level of said α-Klotho polypeptide in said second urine sample is greater than the level of said α-Klotho polypeptide in said first urine sample; and
(d) identifying said anti-senescence treatment as being not effective if the level of said α-Klotho polypeptide in said second urine sample is less than or equal to the level of said α-Klotho polypeptide in said first urine sample.

2. The method of claim 1, wherein said mammal is a human.

3. The method of any one of claims 1-2, wherein said first urine sample is obtained prior to said mammal having been administered said anti-senescence treatment.

4. The method of any one of claims 1-2, wherein said first urine sample is obtained after said mammal has been administered said anti-senescence treatment.

5. A method for assessing efficacy of an anti-senescence treatment, wherein said method comprises detecting a level of an α-Klotho polypeptide in a urine sample obtained from a mammal at least 120 hours after administration of said anti-senescence treatment to said mammal, wherein said anti-senescence treatment is identified as being effective if the level of said α-Klotho polypeptide in said sample is greater than 293.49±115.48 ng of said α-Klotho polypeptide per mg of creatinine present in said urine sample, and wherein said anti-senescence treatment is identified as being ineffective if the level of said α-Klotho polypeptide in said sample is less than 293.49±115.48 ng of said α-Klotho polypeptide per mg of creatinine present in said urine sample.

6. The method of claim 5, wherein said mammal is a human.

7. A method for increasing a level of an α-Klotho polypeptide in a mammal, said method comprising administering a senotherapeutic agent to said mammal.

8. The method of claim 7, wherein said mammal is a human.

9. The method of any one of claims 7-8, wherein said human is identified as being in need of increased α-Klotho polypeptide expression.

10. The method of any one of claims 7-9, wherein said mammal has fibrosis.

11. The method of claim 10, wherein said fibrosis is idiopathic pulmonary fibrosis (IPF).

12. The method of any one of claims 7-11, wherein said senotherapeutic agent is selected from the group consisting of dasatinib, quercetin, navitoclax, A1331852, A1155463, fisetin, luteolin, geldanamycin, tanespimycin, alvespimycin, piperlongumine, panobinostat, FOX04-related peptides, nutlin3a, ruxolitinib, metformin, rapamycin procyanidin C1, SSK1, Prodrug A (JHB75B), 5FURGal, Nav-Gal, PZ15227, PROTAC ARV825, and CD9-Lac/CaCO3/Rapa nanoparticles.

13. The method of any one of claims 7-12, wherein said level of said α-Klotho polypeptide is detected in a urine sample obtained from said mammal.

14. The method of claim 13, wherein said level of said α-Klotho polypeptide is greater than 293.49±115.48 ng of said α-Klotho polypeptide per mg of creatinine present in said urine sample.

15. A method for increasing a level of an α-Klotho polypeptide in a mammal, said method comprising administering an inhibitor of a senescence-associated secretory phenotype (SASP) polypeptide to said mammal.

16. The method of claim 15, wherein said mammal is a human.

17. The method of any one of claims 15-16, wherein said human is identified as being in need of increased α-Klotho polypeptide expression.

18. The method of any one of claims 15-17, wherein said mammal has fibrosis.

19. The method of claim 18, wherein said fibrosis is IPF.

20. The method of any one of claims 15-19, wherein said SASP polypeptide is an activin A polypeptide or an interleukin 1α (IL-1α) polypeptide.

21. The method of any one of claims 15-20, wherein said inhibitor of said SASP polypeptide is a neutralizing antibody.

22. The method of any one of claims 15-21, wherein said level of said α-Klotho polypeptide is detected in a urine sample obtained from said mammal.

23. The method of claim 22, wherein said level of said α-Klotho polypeptide is greater than 293.49±115.48 ng of said α-Klotho polypeptide per mg of creatinine present in said urine sample.

24. The use of a composition comprising a senotherapeutic agent to increase α-Klotho polypeptide expression in a mammal.

25. The use of a composition comprising an inhibitor of a SASP polypeptide to increase α-Klotho polypeptide expression in a mammal.

26. The use of claim 24 or 25, wherein said mammal is a human.

27. The use of claim 26, wherein said human has fibrosis.

28. The use of any one of claims 24-27, wherein said level of said α-Klotho polypeptide is detected in a urine sample obtained from said mammal.

29. The method of claim 28, wherein said level of said α-Klotho polypeptide is greater than 293.49±115.48 ng of said α-Klotho polypeptide per mg of creatinine present in said urine sample.

30. A senotherapeutic agent for use in the preparation of a medicament to increase α-Klotho polypeptide expression in a mammal.

31. A senotherapeutic agent for use in increasing α-Klotho polypeptide expression in a mammal.

32. The senotherapeutic agent of claim 30 or 31, wherein said mammal is a human.

33. The senotherapeutic agent of claim 32, wherein said human has fibrosis.

34. The senotherapeutic agent of any one of claims 30-33, wherein said level of said α-Klotho polypeptide is detected in a urine sample obtained from said mammal.

35. The senotherapeutic agent of claim 34, wherein said level of said α-Klotho polypeptide is greater than 293.49±115.48 ng of said α-Klotho polypeptide per mg of creatinine present in said urine sample.

36. An inhibitor of a SASP polypeptide for use in the preparation of a medicament to increase α-Klotho polypeptide expression in a mammal.

37. An inhibitor of a SASP polypeptide for use in increasing α-Klotho polypeptide expression in a mammal.

38. The inhibitor of a SASP polypeptide of claim 36 or 37, wherein said mammal is a human.

39. The inhibitor of a SASP polypeptide of claim 38, wherein said human has fibrosis.

40. The inhibitor of a SASP polypeptide of any one of claims 36-39, wherein said level of said α-Klotho polypeptide is detected in a urine sample obtained from said mammal.

41. The inhibitor of a SASP polypeptide of claim 40, wherein said level of said α-Klotho polypeptide is greater than 293.49±115.48 ng of said α-Klotho polypeptide per mg of creatinine present in said urine sample.
